SEQUENCE LISTING (1 sequence)

SEQ ID NO: 1 - protein, 34 aa, Artificial Sequence
  Parathyroid Hormone Fragment
SVSEIQLMHNLGKHLNSMERVEWLRKKLQDVHNF

(19) United States
(12) Patent Application Publication
(10) Pub. No.: US 2022/0193202 A1
(43) Pub. Date: Jun. 23, 2022

(54) PARATHYROID HORMONE DERIVATIVE IN DIMERIC FORM OF PARATHYROID HORMONE FRAGMENT AND PHARMACEUTICAL COMPOSITION FOR PREVENTION OR TREATMENT OF BONE DISEASES COMPRISING SAME

(71) Applicants: GACHON UNIVERSITY OF INDUSTRY-ACADEMIC COOPERATION FOUNDATION, Seongnam-si, Gyeonggi-do (KR); GIL MEDICAL CENTER, Incheon (KR)

(72) Inventor: Sihoon LEE, Seoul (KR)

(73) Assignees: GACHON UNIVERSITY OF INDUSTRY-ACADEMIC COOPERATION FOUNDATION, Seongnam-si, Gyeonggi-do (KR); GIL MEDICAL CENTER, Incheon (KR)

(21) Appl. No.: 17/600,923

(22) PCT Filed: Nov. 7, 2019
(86) PCT No.: PCT/KR2019/015078

§ 371 Date: Oct. 1, 2021

(30) Foreign Application Priority Data
Apr. 29, 2019 (KR) 10-2019-0050079

(51) Int. Cl. A61K 38/29; A61P 19/08; C07K 14/635

(52) U.S. Cl. CPC A61K 38/29; C07K 14/635; A61P 19/08

(57) ABSTRACT

The present invention relates to a parathyroid hormone derivative in a dimeric form of a parathyroid hormone fragment and a pharmaceutical composition for the prevention or treatment of bone diseases comprising same. When administered to an animal model, the parathyroid hormone derivative in the dimeric form of the parathyroid hormone according to the present invention significantly increases bone density and enhances osteogenesis and thus can be effectively used for the treatment and prevention of bone diseases.

### TECHNICAL FIELD

The present disclosure relates to a parathyroid hormone derivative in a dimeric form of a parathyroid hormone fragment, and a pharmaceutical composition for prevention or treatment of bone diseases comprising the same.

### BACKGROUND ART

Osteoporosis, a representative metabolic bone disease, is a disease in which bone density is lowered due to a decrease in calcification of bone tissue, and thereby a bone marrow cavity is widened as the disease progresses, leading to bone fracture even though there is a tiny impact.

Osteoporosis has features such as a decrease in bone mass and an abnormality of microstructure. This disease is caused because the balance between the resorption of old bone and the formation of new bone is broken, and the replacement of new bone (called, “bone remodeling”) does not progress smoothly. As a result of this, empty spaces are generated inside bones and the risk of fracture or breakage of a bone increases. Such bone mass is affected by several factors including genetic factors, nutritional intake, hormonal changes, and exercise and differences in lifestyle. The main causes of a decrease in bone mass include old age, lack of exercise, low body weight, smoking, a low-calcium diet, menopause, ovarian resection, inflammation, or the like.

Although there are differences between individuals, in general, bone mass is highest at the age of 14 to 18 and decreases by about 1% per year in old age. In particular, bone loss continues to progress after the age of 30 in the case of women, and when they reach menopause, bone loss progresses rapidly due to hormonal changes. During menopause, osteoclast activity increases due to a decrease in estrogen concentration in the body, leading to a decrease in bone mass.

As such, although there is a difference in degree, osteoporosis is an unavoidable symptom for the elderly, in particular postmenopausal women, and interest in osteoporosis and its therapeutic agents is gradually increasing in developed countries as the population ages. In addition, the market related to the treatment of bone diseases is worth about 130 billion dollars globally, and it is expected to increase further in the future, so global research institutes and pharmaceutical companies are investing a lot in the development of therapeutic agents for bone diseases.

Estrogen has been well-known as a therapeutic agent for osteoporosis, has not yet been verified for its practical efficacy, must be taken throughout one's life, and has a side effect of increasing the incidence of breast cancer or uterine cancer when it is administered for a long period of time. In addition, an efficacy of alendronate which is another therapeutic agent for osteoporosis is not clear, its absorption in the digestive tract is slow, and there is a problem of causing inflammation in the stomach and esophageal mucosa. On the other hand, although calcium agents are effective with few side effects, they are a preventive agent rather than a therapeutic agent. In addition, efficacy and side effects of vitamin D agents such as calcitonin have not yet been sufficiently studied.

Accordingly, the present inventors discovered that a dimer of parathyroid hormone promotes bone formation in fracture animal models in searching for a substance for treating bone diseases by regulating the activity of osteoclasts or bone metabolism, and then completed the present disclosure.

### DISCLOSURE

### Technical Problem

An objective of the present disclosure is to provide a parathyroid hormone derivative in a dimeric form of parathyroid hormone fragments.

Another objective of the present disclosure is to provide a pharmaceutical composition for preventing or treating bone diseases comprising parathyroid hormone derivative in a dimeric form of parathyroid hormone fragments.

### Technical Solution

In order to accomplish the above object, the present disclosure provides a parathyroid hormone derivative in a dimeric form of parathyroid hormone fragments.

The parathyroid hormone fragment may include cysteine at a 25th amino acid position from the N-terminus of its amino acid sequence, and the parathyroid hormone fragment may include a 1^{st} amino acid to a 34^{th} amino acid from the N-terminus of its amino acid sequence. The parathyroid hormone fragment may be an amino acid sequence set forth in SEQ ID NO: 1.

In addition, in order to achieve the above object, the present disclosure provides a pharmaceutical composition for preventing or treating bone diseases comprising a parathyroid hormone derivative.

The bone diseases may be a fracture, osteoporosis, rheumatoid arthritis, osteoarthritis, Paget disease, or metastatic bone cancer.

### Advantageous Effects

As described above, the present disclosure provides useful applicability for preventing or treating bone diseases by administering a parathyroid hormone derivative in a dimeric form of parathyroid hormone fragments to an animal model, thereby increasing bone density and bone formation significantly.

### DESCRIPTION OF DRAWINGS

FIG. 1 is a graph confirming a purity of a prepared dimer, a dimeric [Cys25]PTH(1-34) according to an embodiment of the present disclosure.

FIG. 2 is a graph showing the results of liquid chromatography/mass spectrometry (LC-MS2) of a dimeric [Cys25]PTH(1-34) according to an embodiment of the present disclosure.

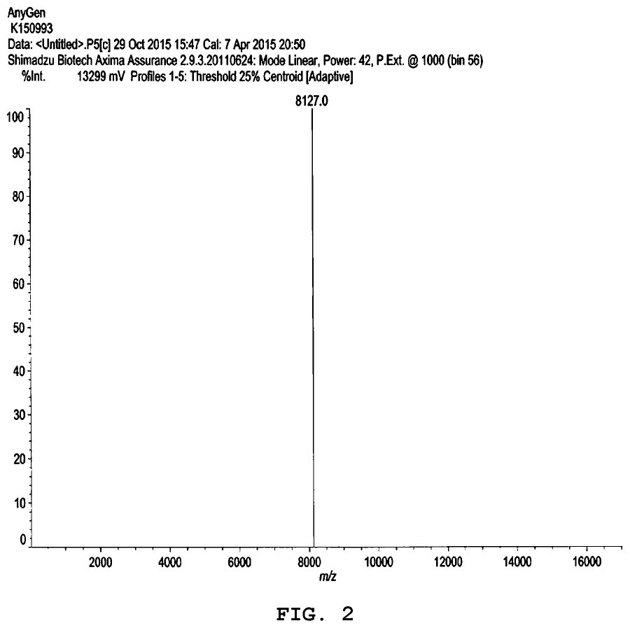

FIG. 3 is a diagram schematically illustrating an experimental procedure for confirming whether a dimeric [Cys25]PTH(1-34) according to an embodiment of the present disclosure exhibits an osteogenic effect in a fracture animal model.

FIG. 4 is an Image Visualization and Infrared Spectroscopy (IVIS) imaging result confirming that a dimeric [Cys25]PTH(1-34) according to an embodiment of the present disclosure exhibits an osteogenic effect in a fracture animal model.

FIG. 5 is a graph showing the result of FIG. 4 according to an embodiment of the present disclosure analyzed as an average of fluorescence flux (photon flux).

FIG. 6 is 3D-micro CT results confirming that a dimeric [Cys25]PTH(1-34) according to an embodiment of the present disclosure exhibits the effect of callus formation in a fracture animal model.

FIG. 7 is a graph showing digitized result of FIG. 6 according to an embodiment of the present disclosure.

FIG. 8 is a graph showing changes in bone formation in the trabecular bone of the femur in a fracture animal model administered with a dimeric [Cys25]PTH(1-34) according to an embodiment of the present disclosure.

### BEST MODE

### Mode for Invention

Hereinafter, the present disclosure will be described in detail.

An embodiment of the present disclosure is a parathyroid hormone derivative in a dimeric form of parathyroid hormone fragments.

The parathyroid hormone fragment may include cysteine at a 25th amino acid position from the N-terminus of its amino acid sequence, and include a 1^{st} amino acid to 34^{th} amino acid from the N-terminus of its amino acid sequence. The parathyroid hormone fragment may be a polypeptide consisting of an amino acid sequence set forth in SEQ ID NO: 1.

In addition, the parathyroid hormone fragment may be a variant of amino acids having a different sequence by deletion, insertion, substitution of amino acid residues, or a combination thereof within a range that does not affect the function of the protein. Exchanges of amino acids in proteins or peptides that do not entirely alter their activity are known in the art. In some cases, it may be modified by phosphorylation, sulfation, acrylation, glycosylation, methylation, or farnesylation. Accordingly, the parathyroid hormone transforming derivative may include a polypeptide having substantially the same amino acid sequence as the polypeptide set forth in SEQ ID NO: 1 has, and variants or fragments thereof. The polypeptide having the substantially the same amino acid sequence may have at least 80% homology with the polypeptide of the present disclosure.

The parathyroid hormone derivative may be a protein in a dimeric form of parathyroid hormone fragments linked by genetic fusion or chemical bond. The term “genetic fusion” refers to a bond formed through genetic expression of a DNA sequence encoding a protein. Meanwhile, the “chemical bond” refers to a bond formed by electrostatic attraction or repulsion generated between atoms constituting a dimer. Examples of the chemical bond may include an ionic bond, a covalent bond, a hydrogen bond, a disulfide bond, and the like.

The parathyroid hormone derivative may include at least one bond between each monomer constituting the dimer, wherein the bond may be formed between any amino acids in the amino acid sequence constituting each monomer. In particular, the bond may be formed by a disulfide bond between cysteines while arginine, which is the 25th amino acid of the parathyroid hormone fragment, is substituted with cysteine. In addition, the dimer may be formed by joining one side or both sides of the carboxy terminus and amino terminus of each monomer together.

Another embodiment of the present disclosure is a pharmaceutical composition for preventing or treating bone diseases, characterized in that it contains the parathyroid derivative.

The bone diseases may be a fracture, osteoporosis, rheumatoid arthritis, osteoarthritis, Paget disease, or metastatic bone cancer.

The pharmaceutical composition for preventing or treating bone diseases may also include carriers, diluents, excipients commonly used in biological agents, or a combination of two or more of them. The pharmaceutical composition for preventing or treating bone diseases may be formulated as an oral formulation or a parenteral formulation. A Solid formulation for oral administration may include tablets, pills, powders, granules, capsules, troches, and the like, and a liquid formulation may include suspensions, internal solutions, emulsions, or syrups. Formulation for parenteral administration may include injectable agents such as sterile aqueous solutions, non-aqueous solutions, suspension solutions, emulsions, and the like.

Hereinafter, the present disclosure will be described in more detail through examples. These examples are only for illustrating the present disclosure, and it will be apparent to those of ordinary skill in the art that the scope of the present disclosure is not to be construed as being limited by these examples.

### Example 1. Preparation of Dimeric [Cys25]PTH

DNA fragment of a parathyroid hormone ([Cys25]PTH(1-34)), wherein a 25th amino acid of PTH(1-34), arginine (Arg) is substituted with cysteine (Cys), was transfected into cells. Mass spectrometry was performed on [Cys25]PTH(1-34) synthesized in the transfected cells. As a result, the mass of the synthesized [Cys25]PTH(1-34) was 8127.0 Da, which was almost the same as the theoretically expected mass of the dimeric [Cys25]PTH(1-34), 8127.4 Da. Therefore, based on the above analysis results, it can be seen that the dimeric form of [Cys25]PTH(1-34) was prepared as a result of transfection of the parathyroid hormone ([Cys25]PTH(1-34)) DNA fragment into cells. (see Table 1

TABLE 1

| | | |
|---|---|---|
| Mass analysis | Instrument | AXIMA CFR Kratos, Shimadzu |
| | MS expected | 8127.4 Da |
| | MS found | 8127.0 Da |

In addition, the prepared dimeric [Cys25]PTH was analyzed by high performance liquid chromatography (HPLC) and liquid chromatography/mass spectrometry (LC-MS2) techniques.

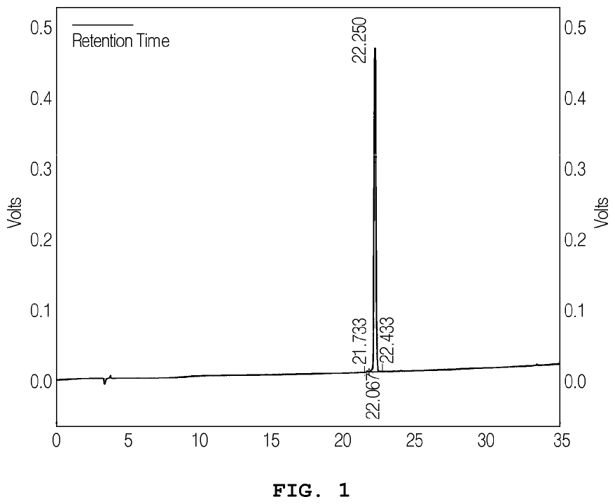

The results as shown in Table 2 and FIG. 1 below confirmed that a purity of prepared dimer was appropriate. That is, Area % in Table 2 indicates the purity of the synthesized product, and Pk # number refers to a constituent material constituting the prepared dimeric synthesized product. The peak shown in FIG. 1 and Pk # data indicate that [Cys25]PTH(1-34) is a dimeric product in the synthesized products. Referring to Table 2, [Cys25]PTH(1-34) dimer, a material corresponding to the Pk #3 had a retention time of 22.250 min as described in the peak of FIG. 1, and the purity (Area %) is 98.5% or more.

TABLE 2
Decector A (230 nm)

| Pk # | retention time | Area | Area % |
|---|---|---|---|
| 1 | 21.733 | 12551 | 0.300 |
| 2 | 22.067 | 33518 | 0.801 |
| 3 | 22.250 | 4122093 | 98.564 |
| 4 | 22.433 | 13972 | 0.334 |
| total | | 4182134 | 100.00 |

Therefore, according to the above experimental results, it was found that a high-purity dimeric form of a parathyroid hormone derivative, [Cys25]PTH(1-34) was synthesized.

### Example. 2 Preparation of Fracture Animal Model

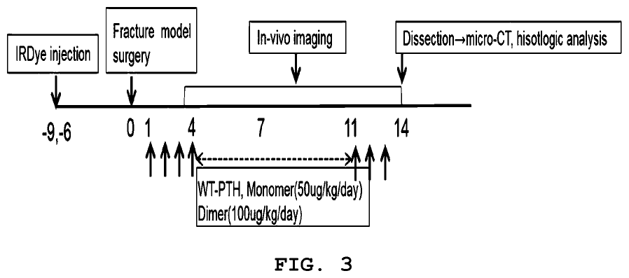

Five 10-week-old C57BL6 male mice were prepared for each group, and fracture model surgery was performed in the following manner (see FIG. 3).

In particular, 9 days before (−D9) and 6 days before (−D6) prior to fracture model surgery (DO) in mice of each group, infrared fluorescent dyes (IR Dyes, Infra-Red dyes) were administered through a tail vein of the mice. Then, the mice were general anesthetized using isoflurane containing oxygen (O2) for fracture model surgery, and a left hind limb was shaved and sterilized for surgery. An incision of about 15 mm was made in the left hind limb in the longitudinal direction, and the tibia was exposed by obtuse-angled resection of the muscle. Then, transverse osteotomy was performed using disc-shaped dental steel bars at the midpoint of the tibia. After repositioning the fracture site, the entire length of a bone marrow lumen was made internally stabilized. After washing it with saline, the skin was sutured with nylon sutures.

### Experimental Example 1. Confirmation of the Osteogenic Promoting Effect of Parathyroid Hormone Derivative, Dimeric [Cys25]PTH(1-34), Using IVIS Imaging Analysis

The following experiment was performed to confirm whether the parathyroid hormone derivatives prepared in Example 1 exhibited an osteogenic effect in a fracture animal model (see FIG. 3).

In particular, the fracture animal models prepared in Example 2 was divided into a control group, PTH(1-34), monomeric [Cys25]PTH(1-34), and dimeric [Cys25]PTH(1-34) administration groups (n=5 animals per group). Each of vehicle, PTH(1-34) and monomeric [Cys25]PTH(1-34) was injected subcutaneously daily at 50 μg/kg for each group from Day 1 to Day 13 after the fracture model surgery was performed. On the other hand, the dimeric [Cys25]PTH(1-34) was injected subcutaneously in an amount of 100 μg/kg to be administered at the same molar ratio.

On Day 4, Day 7, Day 11, and Day 14 after the surgery, fracture sites of mice per each group were photographed and analyzed using IVIS Lumina XR (Caliper Life Sciences, USA) equipment and Living Image software.

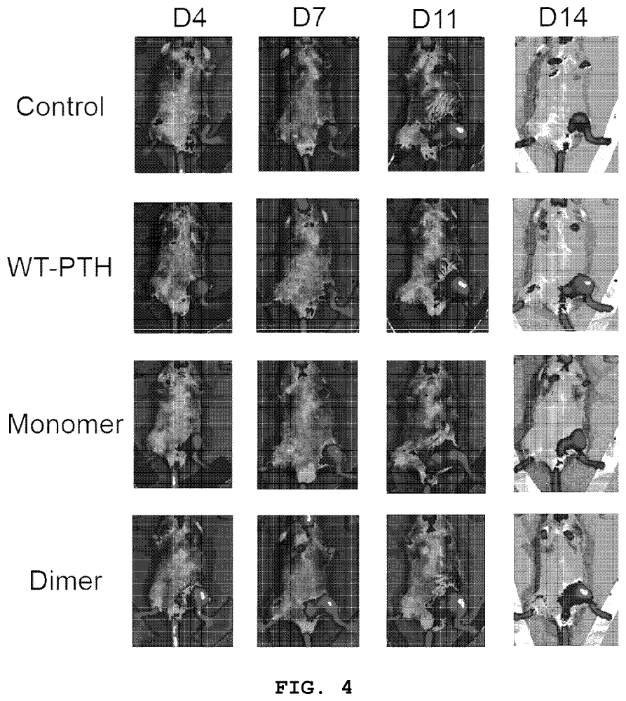

The results as shown in FIG. 4 confirmed that when the dimeric [Cys25]PTH(1-34) was administered, an intensity of fluorescence was increased at the site where the fracture was healing. A degree of intensity of fluorescence indicates that photons are flowing, wherein the more calcium accumulates, the more flow of photons increases, whereby the fluorescence becomes more intense. Therefore, intensifying of the fluorescence means that bone formation increased.

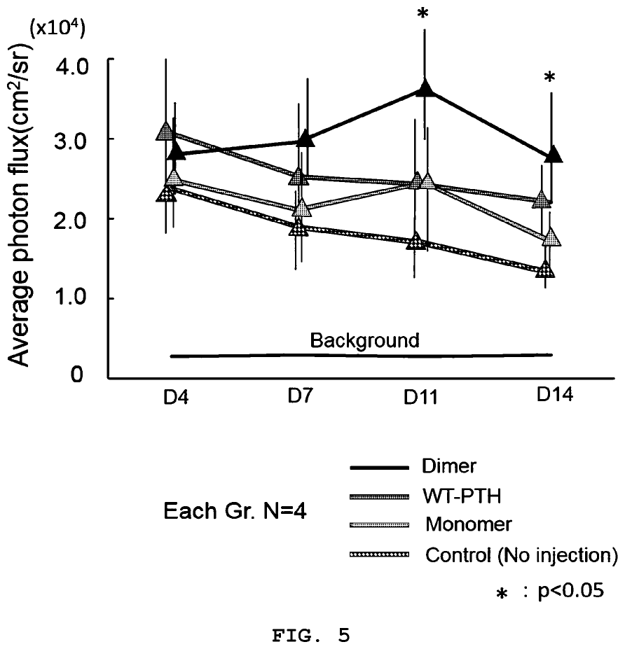

Also, as shown in FIG. 5, it can be found that a result of analyzing the results of FIG. 4 as an average of photon flux indicates that the dimeric [Cys25]PTH(1-34), a parathyroid hormone derivative, significantly increased bone formation.

### Experimental Example 2. Confirmation of Osteogenesis Promoting Effect of Parathyroid Hormone Derivative, Dimeric [Cys25]PTH(1-34), Using 3D-Micro CT Image Analysis

The osteogenesis promoting activity of the parathyroid hormone derivative, the dimeric [Cys25]PTH(1-34), confirmed in Experimental example 1, was reconfirmed by taking 3D-micro CT images.

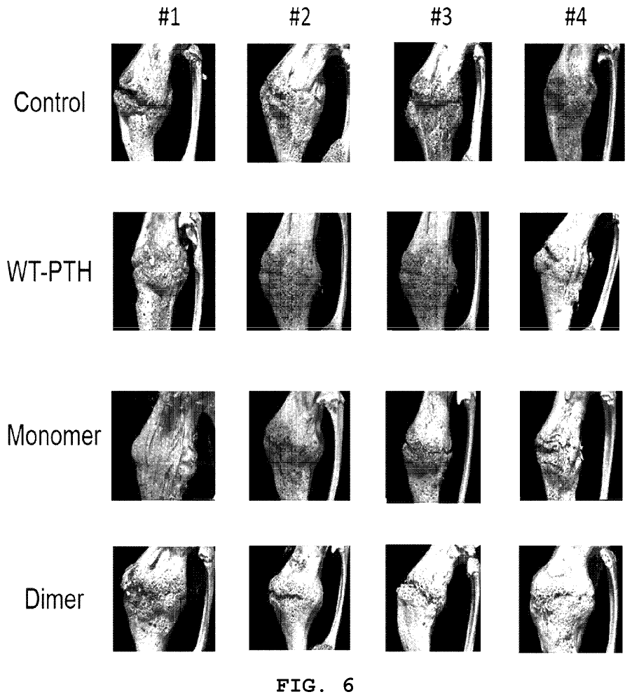

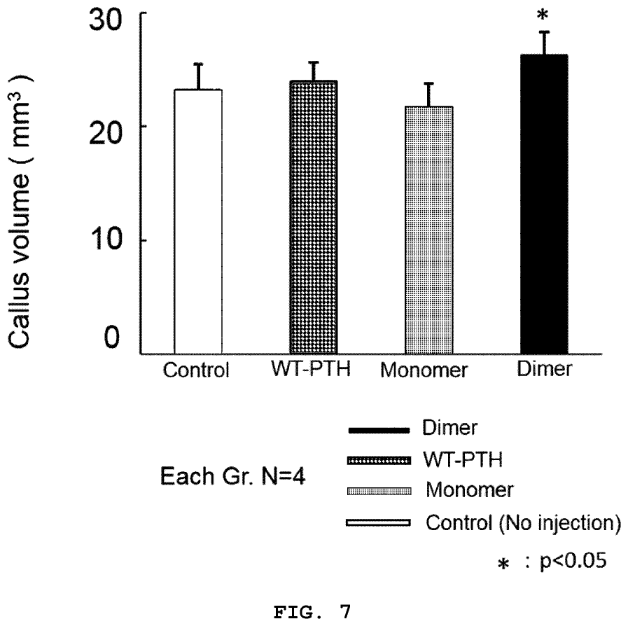

In particular, after the fracture site was photographed on Day 14 in the above experimental example 1, the mice were sacrificed with carbon dioxide (CO2) and incised to collect the shin bones (tibia) including the fracture site. The collected shin bones were imaged and analyzed using 3D-micro CT (Microfocus X-ray CT system SMX-90CT, Shimadzu, Japan) equipment. The result as shown in FIG. 6 indicates that when the dimeric [Cys25]PTH(1-34) was administered, callus formation was promoted in the fracture animal model. In addition, the results converted into numerical values as shown in FIG. 7 confirms that when the dimeric [Cys25]PTH(1-34) is administered, callus volume (mm3) in the fracture animal model is notably increased.

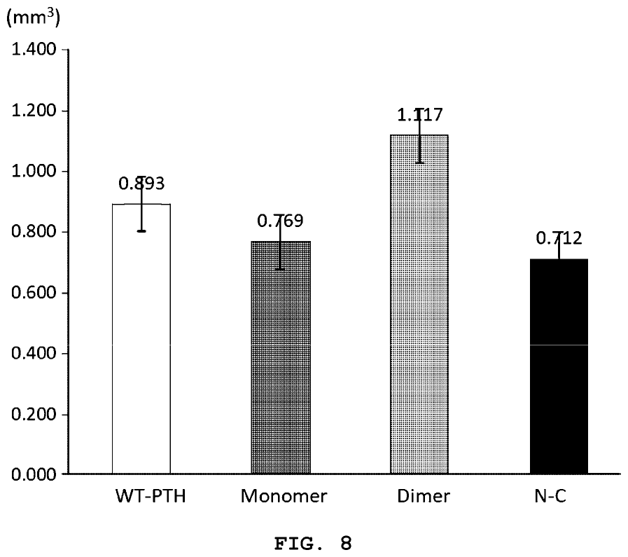

In addition, the femurs of an opposite limb of the animal models were collected and analyzed by micro-CT equipment to evaluate the degree of bone formation in the trabecular bones of the femurs. As a result, as shown in FIG. 8, it was found that the administration of the dimeric [Cys25]PTH(1-34) resulted in the greatest increase in bone formation in the trabecular bones of the mice femurs.

As described above, a particular part of the present disclosure has been described in detail, and the specific description is only a preferred embodiment, and the fact that the scope of the present disclosure is not limited thereby will be clear that to those of ordinary skill in the art. Accordingly, it is intended that the substantial scope of the present disclosure will be defined by the appended claims and their equivalents.

### Free Text of Sequence List

The electronic file is attached.

1. A parathyroid hormone derivative in a dimeric form of parathyroid hormone fragments.

2. The parathyroid hormone derivative according to claim 1, wherein the parathyroid hormone fragment comprises cysteine at the twenty fifth amino acid position from the N-terminus of an amino acid sequence thereof.

3. The parathyroid hormone derivative according to claim 1, wherein the parathyroid hormone fragment comprises the first amino acid to the thirty fourth amino acid from the N-terminus of a amino acid sequence thereof.

4. The parathyroid hormone derivative according to claim 1, wherein the parathyroid hormone fragment is an amino acid sequence set forth in SEQ ID NO: 1.

5. A pharmaceutical composition for preventing or treating bone diseases, the composition comprising the parathyroid hormone derivative according to claim 1.

6. The pharmaceutical composition according to claim 5, wherein the bone diseases are a fracture, osteoporosis, rheumatoid arthritis, osteoarthritis, Paget disease, or metastatic bone cancer.
